(19) United States
(12) Patent Application Publication
(10) Pub. No.: US 2010/0297663 A1
(43) Pub. Date: Nov. 25, 2010

(54) METHODS AND SYSTEMS FOR ASSAYING, MAINTAINING, AND ENHANCING THE ACTIVITY OF THE IMMUNE SYSTEM OF A SUBJECT

(75) Inventors: Brent VAUGHAN, Kearns, UT (US); Shane M. LEFLER, American Fork, UT (US); Richard H. BENNETT, Captain Cook, HI (US); Calvin M. McCAUSLAND, Springville, UT (US); David LISONBEE, Orem, UT (US)

Correspondence Address: Durham, Jones & Pinegar --;Intellectual Property Law Group, P.O. Box 4050, Salt Lake City, UT, 84110, US

(73) Assignee: 4LIFE PATENTS, LLC, Sandy, UT (US)

(21) Appl. No.: 12/774,659

(22) Filed: May 5, 2010

Related U.S. Application Data
(60) Provisional application No. 61/175,740, filed on May 5, 2009.

(51) Int. Cl. G01N 33/53

(52) U.S. Cl. 435/7.1

(57) ABSTRACT

A method for non-invasively assessing a subject's health includes evaluating a state of a subject's immune response. Such a method may include obtaining a salivary sample from the subject and assaying the salivary sample for IgA. The state of a component of the subject's immunity may be evaluated in conjunction with the administration of one or more substances known to elicit a cell-mediated immune response to the subject to determine the effect of the one or more substances on the subject's humoral, or antibody-mediated, immune response. Assay methods may also be used to optimize the dosage of an immune support component to be administered to a particular subject. Systems that include assays for evaluating the state of a subject's immune response and nutraceuticals are also disclosed.

### CROSS-REFERENCE TO RELATED APPLICATION

This application claims the benefit of priority under 35 U.S.C. §119(e)(1) to U.S. Provisional Patent Application 61/175,740, filed May 5, 2009, the disclosure of which hereby incorporated herein, in its entirety, by this reference.

### TECHNICAL FIELD

The present invention relates generally to methods and systems for assessing the immune activity, or immune health, of a subject and, more specifically, to methods and systems for non-invasively assessing the activity of a subject's immune system, including a cell-mediated, or T cell based, component of the subject's immune system; a humoral, or B-cell based or antibody-based, component of the subject's immune system; or both the cell-mediated and the humoral components of the subject's immune system. The present invention includes methods and systems that are based upon an assessment of a subject's salivary IgA to provide an indication of the activity, or health or strength, of one or more components of a subject's immune system. In some more specific embodiments, methods and systems of the present invention may be used to evaluate the effect of one or more nutraceuticals on the one or more components of a subject's immune system.

### BACKGROUND OF RELATED ART

Immunoassays are clinical tests that are typically configured to evaluate a sample from a subject and to provide an indication of whether or not a particular antigen or an antibody with specificity for a particular antigen is present in the sample. When an immunoassay is configured to assay antibodies, the immunoassay may provide some indication of the state of a particular component of the subject's immune system (i.e., the level of activity by a B cell component of the subject's immune system against the specified antigen) at the time the sample was obtained.

Other immune tests have been developed to provide a broader indication of a subject's immune health. One such test is a secretory Immunoglobulin A (SIgA) immunoassay, such as that marketed by Salimetrics of State College, Pa. That test is designed to assay the total amount of SIgA as a class of immunoglobulins, regardless of specificity for any particular antigen(s), produced by a subject.

Immunoglobulin A is a class of antibodies that is commonly found in and on mucosal surfaces. As roughly ninety-five percent (95%) of all infections initially occur at mucosal surfaces, the body's secretion of SIgA onto mucosal surfaces provides a first line of defense against infection. Studies have shown that when SIgA levels decrease, as happens with increased stress, strenuous exercise, and at the beginning of an illness, the incidence of upper respiratory tract infections (URTIs) increases.

An immunoassay for SIgA, such as the Salimetrics test, provides some indication of the ability of a subject's immune system to elicit a primary, antibody-based, immune response against an infection.

### BRIEF DESCRIPTION OF THE FIGURES

FIGS. 1 and 2 are bar graphs depicting the average rates at which SIgA was produced over the course of a study to determine the effects of certain natural supplements, or nutraceuticals, on immune activity, or health, by all of the subjects who participated in the study;

FIG. 3 is a bar graph showing the increase in the rate of SIgA production by each subject who participated in the study from the beginning of the study to the end of the study; and

FIG. 4 is a line graph that illustrates the responsiveness of the immune systems of different groups, or quartiles, of the subjects to the natural supplements.

### SUMMARY

The present invention, in one aspect, includes methods for evaluating the activity, or strength or health, of one or more components (e.g., the cell-mediated, or T cell based, component; the humoral, or B cell-based or antibody-based, component, etc.) of a subject's immune system. As used herein, the terms “activity,” “strength,” and “health” refer to, but are not limited to, immune status (e.g., natural and acquired immune resistance appropriately targeted to internal and external antigens), immune competence (e.g., the ability of the immune system to respond appropriately to an antigenic stimulation, and to unleash an immune response “cascade”), and disease risk.

In various embodiments, the activity, or health, of one or more components of a subject's immune system may be determined by assaying quantifiable indicators of immunity. In some embodiments, a method according to the present invention includes non-invasively obtaining a sample from a subject. In a specific embodiment of such a method, a salivary sample is obtained and assayed for secretory IgA. In an even more specific embodiment, a salivary sample is obtained over a period of time so that a rate at which a subject produces saliva may be determined. The salivary sample may then be assayed for SIgA. The rate at which saliva is produced by the subject may be considered in conjunction with (e.g., multiplied by, etc.) the amount of IgA present in an assayed sample of fixed volume to provide an indicator of the amount of SIgA secreted and/or produced by the subject over time, or of the rate at which the subject produces and/or secretes IgA into the saliva.

In another aspect, the present invention includes methods for evaluating the effect of an immune support substance on at least a part of (e.g., a primary immune response by, a secondary immune response by, a cell-mediated immunity component of, a humoral component of, etc.) the immune system of a subject. In such a method, an aspect of the subject's immune system is assayed before the immune support substance is administered to the subject and at one or more points in time after the immune support substance is administered to the subject.

According to another aspect, the present invention includes methods for supporting immune function. Various embodiments of such a method include evaluating the state of a subject's immune system and, if the subject's immune system is not functioning at desirable or even optimal levels, administering an immune support substance to the subject. The state of a subject's immune system may be evaluated following an event that may compromise the subject's immune system (e.g., during or shortly after an illness, following surgery, during or after a rigorous course of athletic training (or overtraining), etc.). In specific embodiments, the state of the subject's immune system may be evaluated by conducting an antibody assay. In a more specific embodiment, the subject's salivary IgA may be evaluated. More specifically, the total amount of SIgA produced by a subject over a predetermined period of time or a rate at which the subject produces SIgA or excretes SIgA into saliva may be evaluated. Such an assessment may, in some embodiments, include comparison of assayed antibody levels to a corresponding “normal” antibody level for the subject. If relatively low antibody levels are detected, an immune support substance may be administered to the subject. Embodiments of immune support substances that may be administered to the subject include, without limitation, transfer factors; the nanofraction immune modulators disclosed by U.S. patent application Ser. No. 11/855,944, filed on Sep. 14, 2007, and titled “NANOFRACTION IMMUNE MODULATORS, PREPARATIONS AND COMPOSITIONS INCLUDING THE SAME, AND ASSOCIATED METHODS, the description of which is hereby incorporated herein, in its entirety, by this reference; other substances that are known to cause a cell-mediated, or T cell based, immune response by a subject; and combinations of substances that are known to elicit a cell-mediated immune response by a subject. After one or more immune support substances have been administered to the subject, or during an ongoing course of immune support treatment, the immune state of the subject may be reevaluated. In some embodiments, one or more immune support substances may be administered until the immune state of the subject reaches and, optionally, maintains “normal” levels for the subject.

The present invention also includes various embodiments of methods for identifying dosages of immune support components that will maintain or improve the activity of the cell-mediated component and/or the humoral component of a particular subject's immune system and/or that will maintain that activity at a desired level for a prolonged period of time. Such a method may include obtaining a baseline measure of some aspect of the subject's immune system, providing the subject with an initial dosage of at least one immune support substance, and monitoring variation in the aspect of the subject's immune system over time. If the assayed aspect of the subject's immune system substantially returns to baseline levels over time, the dosage of the at least one immune support substance may be increased and the aspect of the subject's immune system again assayed over time. If, instead of substantially returning to baseline levels, the assayed aspect of the subject's immune system remains elevated for a prolonged period of time, the dosage of the immune support substance may be reduced and the assayed aspect of the subject's immune system monitored over time. These processes may be repeated until a minimum or optimum dosage that provides the desired effect (e.g., maintenance of an elevated activity of the cell-mediated and/or humoral component of the subject's immune system, etc.) is identified.

In addition, the present invention includes methods for evaluating the effects of immune support components that are known to elicit a cell-mediated response by a subject's immune system, such as transfer factor and/or nanofraction immune modulators, on the humoral component of a subject's immune system.

Systems for maintaining or enhancing the activity, or strength or health, of a subject's immune system are also within the scope of the present invention. In addition to an assay for evaluating the activity, or strength or health, of the subject's immune system, a system of the present invention includes an immune support substance that is to be administered to a subject. That immune support substance may comprise a natural supplement, or nutraceutical. In some embodiments, the nutraceutical may comprise an immune support composition that includes transfer factor, nanofraction immune modulators, another immune support substance that is believed to support the cell-mediated component of a subject's immune system, or any combination of the foregoing.

Other aspects, as well as the features and advantages of various aspects, of the present invention will become apparent to those of ordinary skill in the art from the ensuing description, the accompanying figures, and the appended claims.

### DETAILED DESCRIPTION

A variety of substances are believed to improve the responsiveness of the immune system of a subject to which they are administered. Some immunity enhancing, or immune support, substances, such as transfer factor and nanofraction immune modulators are naturally produced. These immune support substances may be obtained from animal products, such as colostrum and eggs.

A number of studies have been conducted in an effort to better characterize transfer factor and nanofraction immune modulators. These studies have provided information about the types of cells whose responsiveness or activity may be enhanced by transfer factor or nanofraction immune modulators. Specifically, both transfer factor and nanofraction immune modulators are known to have an effect on cell-mediated immunity, while transfer factors were not previously believed to have any effect on humoral, or B cell mediated or antibody mediated, immunity. In view of the belief that transfer factor is involved in cell mediated immunity, these studies have largely been limited to assays that demonstrate the effects of transfer factor and nanofraction immune modulators on various types of T cells.

To the inventors' knowledge, despite the availability of tests for salivary IgA, the effects of transfer factor, nanofraction immune modulators, and other cell-mediated immune support substances on the body's antibody-based first line of defense—IgA that has been secreted onto mucosal surfaces—had not been evaluated.

In an effort to determine whether immune support substances such as transfer factor and nanofraction molecules had any effect on SIgA production or secretion, the inventors developed a study. That study involved a number of subjects to whom neither transfer factor nor nanofraction immune modulators had recently been administered. Initial SIgA levels from these subjects were determined, a transfer factor and/or nanofraction immune modulator administration regimen was initiated and followed for a set period of time, and SIgA levels were assayed periodically throughout the transfer factor and/or nanofraction immune modulator administration regimen. The details of that study are provided in the EXAMPLE below.

### Example

The activity, or health, of the immune systems of twenty-four (24) subjects, or participants, was studied. More specifically, the affects of two nutraceuticals that are believed to support or enhance immune system health were studied. Even more specifically, the affects of transfer factor and/or nanofraction immune modulators on the activity of the cell mediated components of the immune systems of the twenty-four subjects were evaluated.

The twenty-four (24) subjects were all healthy adults. Those who participated in the entire study remained nominally healthy throughout the study. Initially, five (5) men and nineteen (19) women participated in the study. The average age standard deviation (SD) of the subjects who participated in the study was 33.3±9.7 years. Their average height was 64.5±3.6 inches. Their average weight was 162.9±49 pounds.

None of the twenty-four subjects had consumed any transfer factor-containing product for a period of at least six months before the study. During the study, no immunomodulatory medicines or supplements, other than the nutraceuticals that were evaluated during the study, were administered to any of the twenty-four subjects.

The study was an open-label trial, in which both the researchers and subjects had knowledge of the specific compositions that were administered to each subject during the course of the study. The study was also a cross-over trial without a washout period, in which each subject consumed one product over a first given period of time, then another product over a second give period of time, which immediately followed the first period of time.

In particular, neither of the products used in the study was consumed by any of the subjects during the first week (“Week 1”) of the study. During each day of the second and third weeks of the study (“Week 2” and “Week 3,” respectively), each subject consumed two capsules of TRANSFER FACTOR TRI-FACTOR FORMULA (lot 0805450), available from 4Life Research, LLC, of Sandy, Utah, which provided a daily dose of 600 mg of transfer factor from both bovine and avian sources, as well as a dose of nanofraction immune modulators. In the fourth and fifth weeks of the study (“Week 4” and “Week 5,” respectively), each subject consumed two fluid ounces (2 fl. oz.) (60 ml) of TRANSFER FACTOR RIOVIDA TRI-FACTOR FORMULA (lot ASA08225), available from 4Life Research, every day, which provided each subject with a daily dosage of 1,200 mg of transfer factor from both bovine and avian sources, as well as a dose of nanofraction immune modulators.

Salivary samples were obtained from each subject at the outset of the study (i.e., at the beginning of Week 1, or at “Week 0”), as well as at the end of each week during the study. The same protocol was followed each time salivary samples were obtained. Subjects verified that they had not brushed their teeth for at least forty-five (45) minutes before sample collection, had not consumed any food for at least twenty (20) minutes before sample collection, and had thoroughly rinsed their mouths with water about ten (10) minutes before sample collection.

Each time a salivary sample was obtained, a 5 cm² piece of the flexible, thermoplastic film marketed as PARAFILM M® by Pechiney Plastic Packaging, Inc., of Chicago, Ill., was provided to and chewed by the subject from whom the sample was obtained. The subject used his or her tongue to push all excess saliva (i.e., saliva not merely saturating or wetting mucosal surfaces) into and through a straw and into a 15 ml conical collection tube for a period of five (5) minutes. The total amount of saliva collected from each subject, which was considered to be the volume of saliva produced by that subject during the five minute collection period, was recorded. The newly collected saliva was immediately chilled (by placing the collection tube on ice). The chilled saliva sample was then aliquoted to smaller tubes and frozen at a temperature of −70° C. within four hours of collection.

Twenty-one (21) of the twenty-four (24) subjects participated in the entire study and, thus, completed the study.

Once analysis of the saliva samples was to be performed, frozen saliva samples were allowed to thaw at room temperature. Once thawed, the saliva samples were agitated with a vortex mixer, and then centrifuged at 1,500×g for fifteen (15) minutes to remove particulates.

Following centrifugation, samples of the supernatant were collected and assayed for SIgA using an indirect enzyme-linked immunosorbant assay (ELISA) kit available from Salimetrics of State College, Pa. The protocol that accompanied the Salimetrics SIgA ELISA was followed to determine the amount of SIgA in (25 μl of) each saliva sample, as well as to provide some indication of the rate at which each subject secreted and/or produced SIgA at the time the tested saliva sample was obtained.

Specifically, two 25 μl aliquots of each sample were tested. Optical density measurements for the two aliquots from each sample were obtained in a manner known in the art, and compared with the optical densities of various references to determine the amount of SIgA (in μg) in each aliquot. These values were then used to determine the amount of SIgA in the entire saliva sample, and that result was divided by five (5) minutes to determine the rate (in μg/min.) at which SIgA was secreted into the saliva over the period of time up until that sample was obtained.

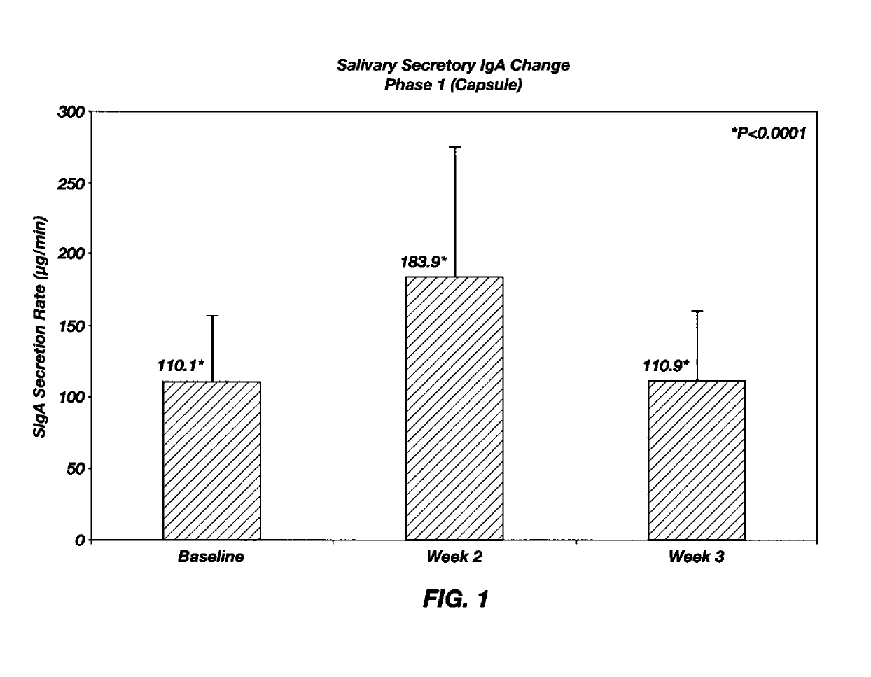

The data that corresponded to the samples obtained at the outset of the study (i.e., at the beginning of Week 1, or at Week 0) and at the end of Week 1 (see FIG. 1) provided a baseline SIgA concentration range of 60 μg/ml to 288 μg/ml and an average±SD SIgA baseline concentration of 125±51 μg/ml. The rates at which SIgA was secreted, or produced, by the subjects who participated in the study during the first two sample periods (i.e., at the beginning and end of Week 1) were also averaged to provide a baseline secretion rate of 110.1 μg/min.

Analysis of variance (ANOVA) data obtained from the test results of all of the samples obtained at the ends of Week 2 and Week 3, which is a referred to herein as a “first phase” of the study, during which two capsules of TRANSFER FACTOR TRI-FACTOR FORMULA were administered daily to each subject, provided an F-distribution (F) of 9.74 and a p-value (p) of 0.0002). The test results of all of the samples obtained at the ends of Weeks 4 and 5, which is referred to herein as a “second phase” of the study, during which two fluid ounces (60 ml) of TRANSFER FACTOR RIOVIDA TRI-FACTOR FORMULA was administered to each subject on a daily basis, provided the following ANOVA data: F=11.35; p=0.00006. These data were deemed to be highly statistically significant.

In view of the high statistical significance of the ANOVA data from the first and second phases, T-tests were performed to determine whether the data from each time point (i.e., Week 2, Week 3, Week 4, Week 5) of the study were statistically different from one another.

As illustrated by FIG. 1, following Week 2, after only one week of receiving transfer factor on a daily basis, about ninety five percent (95%) of the subjects (i.e., twenty (20) of the twenty-one (21) subjects who completed the study) exhibited increased salivary SIgA secretion and, thus, increased SIgA production. For the entire group, the absolute SIgA concentration increased, on average, by about 50 μg/ml, from about 125 μg/ml to about 175 μg/ml, which represents an increase of about thirty-nine percent (39%). The average rate of SIgA secretion for the group increased from 110.1 μg/min. to 183.9 μg/min., for an increase of 73.8 μg/min., or about sixty-seven percent (67%) (p<0.001) over the baseline secretion rate.

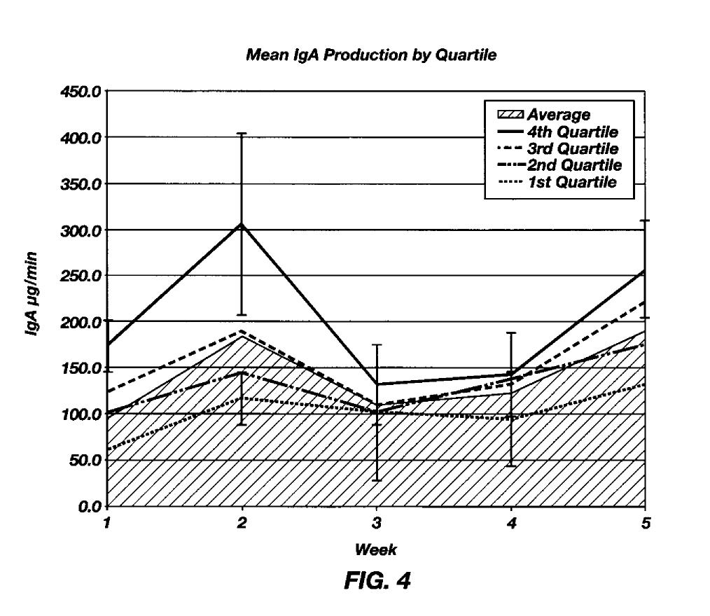

At the end of Week 3, as shown in FIG. 1, the average concentration of SIgA for the group fell to about 102 μg/ml. The average rate at which SIgA was secreted by the subjects, on average, was only 110.9 μg/min., or about the same as the baseline secretion rate. This general decrease in SIgA secretion and production to near baseline rates during the second week of TRANSFER FACTOR TRI-FACTOR FORMULA administration was also observed on a smaller scale, in each of four subgroups, or quartiles, of the twenty-one (21) subjects, as shown by FIG. 4. The decrease in the rate of SIgA secretion and production may have been due to the homeostasis of the immune systems of the tested subjects, which would indicate that, at the time of the study, the subjects already had healthy immune systems that were able to rebalance after having been exposed to transfer factor and/or nanofraction immune modulators for a week or more.

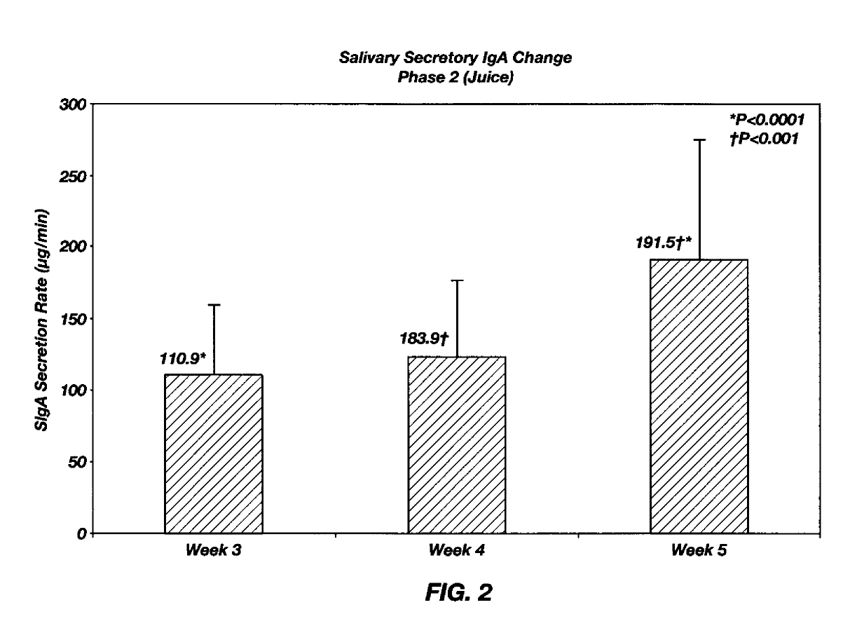

As depicted by FIG. 2, the SIgA values increased again during the second phase of the study. While only slight increases in SIgA concentration and SIgA secretion rate averages were observed from the end of Week 3 to the end of Week 4, by the end of Week 5, the increases in the average SIgA concentration and the average SIgA secretion rate were significant. At the end of Week 5, the average concentration of SIgA in the subjects' saliva had increased to about 49 μg/ml, an increase of about twenty-eight percent (28%) (p<0.001) over the baseline concentration, while the average rate at which SIgA was secreted into the subjects' saliva had increased to 191.5 μg/min., an increase of 73.9% (p<0.001) over the baseline secretion rate. Notably, the dosage of transfer factor was doubled from the first phase of the study to the second phase of the study. The increase in dosage may have eventually (after about a week of continued administration) been sufficient to overcome the homeostatic control of the subjects' healthy immune systems, and may provide some insight as to a dosage of transfer factor that may enable a subject's immune system to produce and secrete SIgA at consistently high (i.e., greater than normal) levels (for that subject).

The averages for each week of the study are reproduced in the following table:

TABLE
Salivary SIgA Summary Data

| | | | Week | Week | Week | Week | Week |
|---|---|---|---|---|---|---|---|
| | Baseline | Week 0 | 1 | 2 | 3 | 4 | 5 |
| Concentration | 125.7 | 149.0 | 102.5 | 175.3 | 102.3 | 111.8 | 175.1 |
| (μg/ml) | | | | | | | |
| Secretion Rate | 110.1 | 124.0 | 96.2 | 183.9 | 110.9 | 123.3 | 191.5 |
| (μg/min.) | | | | | | | |

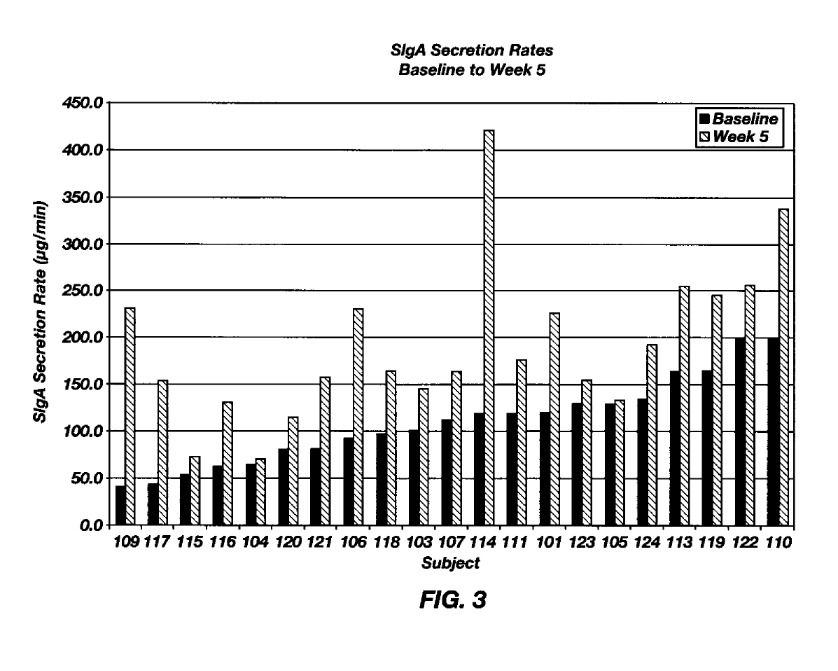

As depicted by FIG. 3, between the outset of the study (i.e., at Week 0) and the end of the study (i.e., at the end of Week 5), the rate at which every one of the subjects who participated in the study produced SIgA increased.

Referring now to FIG. 4, the data was separated into quartiles, with the first quartile including data from subjects who exhibited the lowest initial rate of SIgA secretion and the fourth quartile including data from subjects who exhibited the highest initial SIgA secretion rate. As the graph of FIG. 4 demonstrates, from Week 1 to Week 2 and again from Week 4 to Week 5, the immune systems of subjects in the fourth quartile (i.e., subjects who exhibited the highest initial SIgA secretion rates) were more responsive to transfer factor and/or nanofraction immune modulators than the immune systems of subjects in the first quartile (i.e., subjects who exhibited the lowest initial SIgA secretion rates). Over the entire course of the study, however, the subjects in the first quartile exhibited the highest overall increase in SIgA production and secretion.

FIG. 4 also shows that the SIgA secretion rates of subjects of the second and third quartiles (i.e., subjects who exhibited median initial SIgA secretion rates) closely followed the average SIgA secretion rates for the entire group.

From these results, it is apparent that immune support substances such as transfer factor and nanofraction immune modulators increase the rate at which SIgA is produced and secreted. At least two conclusions can be drawn from this data: (1) an evaluation of SIgA production and/or secretion provides some indication of the activity, or strength or health, of the cell-mediated component of a subject's immune system; and (2) immune support substances that were previously believed to affect cell-mediated immunity without affecting antibody-mediated immunity, such as transfer factor and nanofraction immune modulators, may also enlist the humoral component of a subject's immune system.

In one aspect, the present invention includes methods for evaluating the activity, or strength or health, of at least one of the cell-mediated component and the humoral component of a subject's immune system. In some embodiments, antibody levels may be assayed to provide an indicator of the activity, or strength or health, of the cell-mediated component and/or humoral component of a subject's immune system. Such a method may include the general quantification of antibody levels (i.e., without assessing any antigen specificity).

One embodiment of such a method includes an assay for SIgA produced and/or secreted by the subject. In a more specific embodiment, SIgA levels in the subject's saliva are assayed. In an even more specific embodiment, along with some quantification of SIgA levels in a fixed amount of saliva, the rate at which rate at which the subject produces saliva and, thus, the rate at which SIgA is secreted into the subject's saliva, are calculated. Each of these acts may be effected in the manner described in the EXAMPLE above, or in any other suitable manner.

The present invention also includes methods for evaluating the effect of a substance of interest on at least one of the cell-mediated component and the humoral component of a subject's immune system. In various embodiments, the effect of an immune support substance, such as transfer factor or nanofraction immune modulators, that is known to elicit a cell-mediated immune response by a subject on one or both of the cell-mediated and humoral components of a subject's immune system may be evaluated. An evaluation of the effect of a substance of interest on the immune system of a subject may be conducted under a variety of circumstances, including, but not limited to, with relatively healthy subjects, with subjects whose immune systems are believed to be compromised, with subjects who are subjected to or have recently been subjected to intense physical activity (e.g., athletes in training or who have recently completed training, etc.), with subjects who are subjected to or have recently been subjected to intense mental or emotional stress, and with subjects who have been subjected to conditions that may otherwise affect their immune status.

In various embodiments, an initial assessment of the activity of a subject's immune system may be made before the substance of interest is administered to the subject. The initial assessment may include a single evaluation or a series of evaluations over a predetermined period of time. This evaluation (or these evaluations) provides baseline data, or a baseline value, with which data from one or more subsequent evaluations will be compared.

In some embodiments, the initial assessment may include an evaluation of the cell-mediated component of the subject's immune system. In other embodiments, the initial assessment may include an evaluation of the humoral component of the subject's immune system. As a specific example, either of the foregoing embodiments may include at least one evaluation using an immunoassay for antibodies produced by the subject. More specifically, at least a part of the initial assessment may be made by assaying SIgA. Even more specifically, the initial assessment may involve the use of a salivary SIgA test that includes an analysis of a total amount of SIgA produced or secreted by the subject, regardless of antibody specificity, such as with the SIgA test available from Salimetrics.

Once an initial assessment has been made, a substance of interest may be administered to the subject in any suitable manner. A variety of substances of interest that may affect one or both of the cell-mediated component and the humoral component of a subject's immune system may be evaluated in accordance with a method of the present invention. In various embodiments, the substance of interest may include a natural supplement, or nutraceutical. In more specific embodiments, the effects of nutraceuticals that include transfer factor, nanofraction immune modulators, or a combination of transfer factor and nanofraction immune modulators may be evaluated. Various embodiments of compositions including one or both of these ingredients are available from 4Life Research, LLC, of Sandy, Utah.

In some embodiments, the subject may receive a single administration of the substance of interest. In other embodiments, the substance of interest may be administered periodically over time. In still other embodiments, administration of the substance of interest may occur on an as-needed basis (e.g., in response to a certain event, such as the onset of cold or flu symptoms, during or after vigorous physical activity, etc.). The manner in which the substance of interest is administered may be consistent with prescribed or otherwise predetermined instructions for using the substance of interest.

After the substance of interest has been administered to the subject at least once, the activity of the assayed aspect (e.g., cell-mediated component, humoral component, etc.) of the subject's immune system may again be assessed. In embodiments where the substance of interest is administered to the subject repeatedly (e.g., periodically, etc.) over time, such assessment may be effected once or more during the period of time over which the substance of interest is administered. For the sake of simplicity, each assessment that occurs after administration of a substance of interest is referred to herein as a “subsequent assessment.”

In some embodiments, each subsequent assessment of the activity of the assayed aspect of the subject's immune system may be effected by the same means (e.g., the same type of assay, etc.) and in accordance with the same procedure as that used in the initial assessment. Thus, various embodiments of a method for evaluating the effect of a substance of interest on the assayed aspect of a subject's immune system may include an immunoassay for antibodies, such as SIgA, produced by the subject (e.g., a salivary test in which a rate at which SIgA is produced or secreted is determined, etc.).

Each subsequent assessment may include a single evaluation or a series of evaluations over a predetermined period of time. The data from each subsequent assessment is compared to the baseline data to provide some indication as to the effect of the substance of interest on the assayed aspect of the subject's immune system.

The data from a subsequent assessment may also be compared with data from another subsequent assessment. Such a comparison may provide useful information, such as the effect of the substance of interest on the assayed aspect of the subject's immune system over time, the effectiveness of continued administration of the substance of interest, and the like. Evaluating data from various assessments may also be used to tailor the manner in which a substance of interest is administered to a particular subject (e.g., dosage, regularity, etc.).

The present invention also includes methods for identifying dosages of immune support components that will maintain or improve the activity of one or both of the cell-mediated component and the humoral component of a subject's immune system and that will maintain such elevated activity for a prolonged period of time. Such a method may include obtaining a baseline measure of some aspect of the subject's immune system.

In some embodiments, a baseline measure of the activity of a cell-mediated component and/or a humoral component of the subject's immune system is obtained. In a specific embodiment, a baseline measure of the concentration of salivary SIgA in the subject's saliva and/or a baseline measure of the rate at which the subject secretes SIgA into his or her saliva or the rate at which the subject produces SIgA may be assayed, such as by use of the assay and protocol described in the EXAMPLE above.

Once a baseline measure of immune activity has been obtained, the subject may be provided with an initial dosage regimen (e.g., a set daily dosage, etc.) of at least one immune support substance. The activity of the subject's immune system may be monitored periodically (e.g., at the end of each week, etc.) during the initial dosage regimen. If the assayed aspect of the subject's immune system substantially returns to baseline levels (e.g., within two or three weeks of beginning the initial dosage regimen, at any point while the subject continues the initial dosage regimen, etc.), the subject's treatment regimen may be altered to provide the subject with an increased dosage of the immune support substance used, and assaying continued. If necessary, this process of increasing the dosage may be continued until the activity of assayed aspect of the subject's immune system substantially consistently remains at desirable (e.g., elevated, etc.) levels (e.g., for more than two weeks, more than three weeks, more than four weeks, etc.).

If, instead of substantially returning to baseline levels during the initial dosage regimen, the assayed aspect of the subject's immune system remains elevated for a prolonged period of time (e.g., more than two weeks, more than three weeks, more than four weeks, etc.), the dosage of the immune support substance may be reduced and the assayed aspect of the subject's immune system monitored over time until a minimum or optimum dosage that continues to provide the desired effect (e.g., maintenance of a normal activity or an elevated activity of the cell-mediated component of the subject's immune system, maintenance of a normal activity or elevated activity by a humoral component of the subject's immune system, etc.) is identified.

Additionally, the present invention includes methods for supporting immune function. Various embodiments of such a method include evaluating the state of a subject's immune system and, if the subject's immune system is not functioning or may not be functioning at desirable or even optimal levels, administering an immune support substance to the subject. The state of a subject's immune system may be evaluated following an event that may compromise the subject's immune system (e.g., during or shortly after an illness, following surgery, during or after a rigorous course of athletic training (or overtraining), etc.). In specific embodiments, the state of the subject's immune system may be evaluated by conducting an antibody assay. In a more specific embodiment, the subject's salivary IgA may be evaluated. More specifically, the total amount of SIgA produced by a subject over a predetermined period of time or a rate at which the subject produces SIgA or excretes SIgA into saliva may be evaluated. Such an assessment may, in some embodiments, include comparison of assayed antibody levels to a corresponding “normal” antibody level for the subject. If relatively low antibody levels are detected, an immune support substance may be administered to the subject. Embodiments of immune support substances that may be administered to the subject include, without limitation, transfer factors, nanofraction immune modulators, other substances that are known to cause a cell-mediated immune response by a subject, and combinations of substances that are known to elicit a cell-mediated immune response by a subject. After one or more immune support substances have been administered to the subject, or during an ongoing course of immune support treatment, the immune state of the subject may be reevaluated. In some embodiments, one or more immune support substances may be administered until the immune state of the subject reaches and, optionally, maintains “normal” levels for the subject.

In addition to the methods that have been described, the present invention includes systems for maintaining or even improving the activity, or health, of one or both of the cell-mediated component and the humoral component of a subject's immune system. An embodiment of such a system includes an immune support component and an assay for assessing the activity, or health of the cell-mediated component and/or the humoral component of the subject's immune system.

The immune support component of a system of the present invention may include any substance that is known or believed to support immune function. In some embodiments, an immune support component may comprise substances (e.g., natural supplements, or nutraceuticals, etc.) that may increase activity of one or both of the cell-mediated component and the humoral component of a subject's immune system. In particular, an immune support component that comprises transfer factor, nanofraction immune modulators, or some combination of transfer factor and nanofraction immune modulators. In other embodiments, the immune support component may comprise one or more plant-based substances that are believed to support immune function.

In embodiments where the system maintains or improves cell-mediated immunity, the assay may comprise any test that provides some indication of the activity, or strength or health, of the cell-mediated component of a subject's immune system. Without limiting the scope of the present invention, an immunoassay that non-specifically quantifies antibodies from a sample that has been obtained from the subject may be used as the assay in a system that incorporates teachings of the present invention. A SIgA test is an example of such an immunoassay. In a more specific embodiment, a salivary SIgA immunoassay, such as that available from Salimetrics, may be used in a system that embodies teachings of the present invention. While the Salimetrics salivary SIgA immunoassay is configured to be used in a clinical laboratory, a system that incorporates teachings of the present invention may alternatively include a test that may be used and evaluated by a lay person.

Embodiments of systems that are configured to maintain or improve humoral immunity may also include assays for antibodies, such as total SIgA assays, assays for total amounts of other types of antibodies (e.g., antibodies present in blood or plasma (IgG, IgM), etc.), assays for antigen-specific antibodies, and the like.

In embodiments where the assay is to be conducted in a laboratory, a system of the present invention may also include various laboratory apparatuses for effecting the method. Such apparatuses may include, but are certainly not limited to, pipettes, freezers, centrifuges, incubators, optical monitoring apparatus (e.g., 96 well plate readers, etc.), and the like.

Although the foregoing description contains many specifics, these should not be construed as limiting the scope of the present invention, but merely as providing illustrations of some of the presently preferred embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions and modifications to the invention as disclosed herein which fall within the meaning and scope of the claims are to be embraced thereby.

What is claimed:

1. A method for evaluating a state of a subject's secondary immune response, comprising:
obtaining a sample of saliva from the subject;
assaying salivary immunoglobulin in the sample of saliva; and
comparing an assayed salivary immunoglobulin to a standard salivary immunoglobulin.

2. The method of claim 1, wherein assaying comprises assaying a rate at which salivary immunoglobulin is released into the subject's saliva.

3. The method of claim 1, wherein comparing comprises comparing the assayed salivary immunoglobulin to a control.

4. The method of claim 1, further comprising:
administering a substance of interest to the subject before obtaining the sample.

5. The method of claim 1, wherein assaying salivary immunoglobulin comprises assaying secreted IgA in the saliva.

6. The method of claim 5, wherein comparing comprises comparing the assayed salivary immunoglobulin to a baseline established for the subject before administering the substance of interest to the subject.

7. The method of claim 6, wherein:
administering the substance of interest comprises administering an initial dosage of an immune support substance; and
comparing comprises determining whether the initial dosage of the immune support substance maintains a desired concentration, rate of secretion, or rate of production of the salivary immunoglobulin for at least a predetermined period of time.

8. The method of claim 7, further comprising:
administering an increased dosage of the immune support substance to the subject if the initial dosage does not maintain the desired concentration, rate of secretion, or rate of production of the salivary immunoglobulin for at least the predetermined period of time.

9. The method of claim 8, further comprising:
repeating the obtaining, assaying, and comparing at least once to determine whether the increased dosage of the immune support substance maintains a desired concentration, rate of secretion, or rate of production of the salivary immunoglobulin for at least a predetermined period of time.

10. A method for evaluating an effect of a substance on a subject's secondary immune response, comprising:
evaluating a state of the subject's secondary immune response before administering the substance to the subject, including:
obtaining a sample of saliva from the subject;
assaying salivary immunoglobulin in the sample of saliva; and
comparing an assayed salivary immunoglobulin to a standard immunoglobulin;
administering the substance to the subject; and
evaluating a state of the subject's secondary immune response after administering the substance to the subject, including:
obtaining a sample of saliva from the subject;
assaying salivary immunoglobulin in the sample of saliva; and
comparing an assayed salivary immunoglobulin to a standard immunoglobulin.

11. The method of claim 10, wherein administering the substance to the subject includes administering at least one of transfer factor an a nanofraction immune modulator to the subject.

12. The method of claim 11, wherein administering is effected for a predetermined period of time.

13. The method of claim 12, wherein administering is effected at least once daily for at least one week.

14. The method of claim 13, wherein administering is effected in accordance with recommendations corresponding to a product including at least one of the transfer factor and the nanofraction immune modulator for at least one week.

15. The method of claim 11, wherein administering comprises administering at least one of the transfer factor and the nanofraction immune modulator over a time period during which no other substance that will cause the subject to elicit the secondary immune response.

16. A system for administering a natural supplement to a subject, comprising:
an assay for an immune indicator;
a natural supplement.

17. The system of claim 16, wherein the natural supplement comprises transfer factor.

18. The system of claim 16, wherein the natural supplement comprises at least one nanofraction immune modulator.

19. The system of claim 17, wherein the assay comprises an assay for a non-invasively obtained immune indicator.

20. The system of claim 19, wherein the non-invasively obtained immune indicator comprises a salivary immune indicator.

21. The system of claim 20, wherein the salivary immune indicator comprises secreted IgA.

22. A method for evaluating an effect of a substance that is known to elicit a cell-mediated immune response on a humoral component of a subject's immune system, comprising:
obtaining at least one baseline sample from a subject;
administering at least one substance known to elicit a cell-mediated immune response to the subject after obtaining the at least one baseline sample;
obtaining at least one subsequent sample from the subject after administering the at least one substance to the subject;
assaying the at least one baseline sample and the at least one subsequent sample to determine a baseline antibody level and at least one subsequent antibody level; and
comparing the baseline antibody level to the at least one subsequent antibody level.

23. The method of claim 22, further comprising:
preventing the subject from consuming any other substance known to elicit an immune response between obtaining the at least one baseline sample and obtaining the at least one subsequent sample.

24. The method of claim 22, wherein obtaining the at least one baseline sample and obtaining the at least one subsequent sample comprise obtaining salivary samples from the subject.

25. The method of claim 24, wherein assaying the at least one baseline sample and the at least one salivary sample comprise determining a baseline level of SIgA and at least one subsequent level of SIgA.
